(19) United States
(12) Patent Application Publication
(10) Pub. No.: US 2021/0183504 A1
(43) Pub. Date: Jun. 17, 2021

(54) PATIENT BED EXIT PREDICTION

(71) Applicant: Hill-Rom Services, Inc., Batesville, IN (US)

(72) Inventors: Eric D. Agdeppa, Cary, NC (US); Michael Scott Hood, Batesville, IN (US); Eugene Urrutia, Apex, NC (US)

(21) Appl. No.: 17/110,892

(22) Filed: Dec. 3, 2020

Related U.S. Application Data
(60) Provisional application No. 62/949,091, filed on Dec. 17, 2019.

(51) Int. Cl. G16H 40/20; G16H 40/67; G16H 10/60; A61B 5/11; G06K 19/07; G06N 20/00

(52) U.S. Cl. CPC G16H 40/20; G16H 40/67; G06N 20/00; A61B 5/1115; G06K 19/0723; G16H 10/60

(57) ABSTRACT

Systems and methods for predicting patient bed exit utilize motion sensors to determine when a patient is removing covers from the patient's bed. Sensors are used to detect movement of a patient's feet, the covers, or both. The sensors could be one or more of RFID sensors (tags), infrared motion detection, video motion detection, accelerometers, and load sensors in the bed. An algorithm generated from training data obtained in controlled experiments is used to analyze the sensor information to determine when patient movements indicate that a blanket or covers are being removed by a patient in a bed. When such patient movements are detected, an alert can be issued to caregivers through a call system so that the caregiver is notified that a patient at risk for falling needs assistance in getting out of bed.

### BACKGROUND

Patients in care facilities, such as hospitals, clinics, nursing homes and the like, are often in compromised medical conditions. Injuries sustained by patients due to falls in care facilities result in significant healthcare costs. In an effort to prevent such injuries, various protocols are implemented to mitigate the risks. For example, patients who are likely to fall when moving unassisted may be identified as being a higher risk, and certain protocols may be implemented to reduce the opportunity for the patients to move about unassisted. However, some patients will attempt to get out of bed without assistance, despite receiving instructions to wait for a caregiver. This results in increased fall risk for those patients.

### SUMMARY

Embodiments of the disclosure are directed to predicting exits from patient support systems in order to mitigate injuries associated with patient falls. Sensors embedded in covers of the patient support system and/or attached to the patient detect movement indicative of the patient removing the covers in preparation to exit the patient support system. Alerts to caregivers can help mitigate falls from patients exiting the patient support system unassisted.

In one aspect, a method of predicting exit of a patient support system comprises: establishing a connection between a patient monitoring computing device and at least one radio frequency identification (RFID) reader positioned proximate the patient support system; establishing a connection between the at least one RFID reader and at least one RFID sensor associated with one or more of a blanket, a sock, a bracelet, and an anklet placed on a patient in the patient support system; monitoring, with the patient monitoring computing device, movement on the patient support system using data from the RFID reader, the data indicating a distance between the at least one RFID sensor and the at least one RFID reader; and determining when the data indicates that the patient is exiting the patient support structure.

In another aspect, a system for monitoring patient movements on a bed comprises: a bed configured to support a patient while under medical care; at least one RFID reader positioned proximate the bed; two or more RFID sensors embedded in covers configured to cover a patient on the bed; and a patient monitoring computing device comprising a processor and a memory comprising instructions. When the instructions are executed, the processor operates a patient monitoring system configured to perform a series of operations comprising: establishing a connection between the patient monitoring computing device and the at least one RFID reader; establishing a connection between the at least one RFID reader and the two or more RFID sensors; associating the RFID sensors with a patient at the patient monitoring computing device; monitoring patient movements on the bed based on signals from the RFID reader measuring a distance between the two or more RFID sensors and the at least one RFID reader; detecting patient movements indicating that the patient is exiting the bed; and issuing an alert to a caregiver call system.

In yet another aspect, one or more computer-readable media having computer-executable instructions embodied thereon that, when executed by one or more computing devices, cause the computing devices to: establish a connection between a patient monitoring computing device and at least one radio frequency identification (RFID) reader positioned proximate a patient bed; establish a connection between the at least one RFID reader and at least two RFID transponders embedded in one or more of a blanket and a sock placed on a patient in the patient bed; associate the at least one RFID transponder with the patient at a patient monitoring computing device; monitor, with the patient monitoring computing device, patient movements on the bed based on signals from the RFID reader measuring a distance between the RFID transponders and the at least one RFID reader; detect patient movements indicating that the patient is going to exit the bed, the patient movements being determined based on the speed at which the distance between the two or more RFID sensors and the at least one RFID reader changes; and issue an alert to a caregiver call system.

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 is a schematic diagram illustrating an example system for predicting patient bed exit.

FIG. 2 is a detailed schematic diagram illustrating the patient monitoring system of FIG. 1.

FIG. 3 is a flow chart illustrating an example method of monitoring a patient to mitigate a risk of falling.

FIG. 4 is a flow chart illustrating an example method of setting up a patient monitoring system with patient movement detecting devices.

FIG. 5 is a flow chart illustrating an example method of monitoring a patient to mitigate a risk of falling.

FIG. 6 is a block diagram illustrating example components of a computing device usable in the system of FIG. 1.

FIG. 7 is a schematic diagram illustrating an example implementation of the system of FIG. 1.

FIG. 8 is a schematic diagram illustrating alternative example implementations of the system of FIG. 1.

### DETAILED DESCRIPTION

The present disclosure is directed to systems and methods for predicting when a patient will exit a patient support system, such as a bed, chair, lift, surgical table, etc. (reference will be made to a “bed” herein for ease of description). Many patients in a hospital are prone to falling due to age, medications, surgery, and medical equipment. In order to mitigate fall risk, a caregiver may assist at-risk patients to exit the bed and walk. However, patients often do not wait for a caregiver and instead leave the bed without assistance.

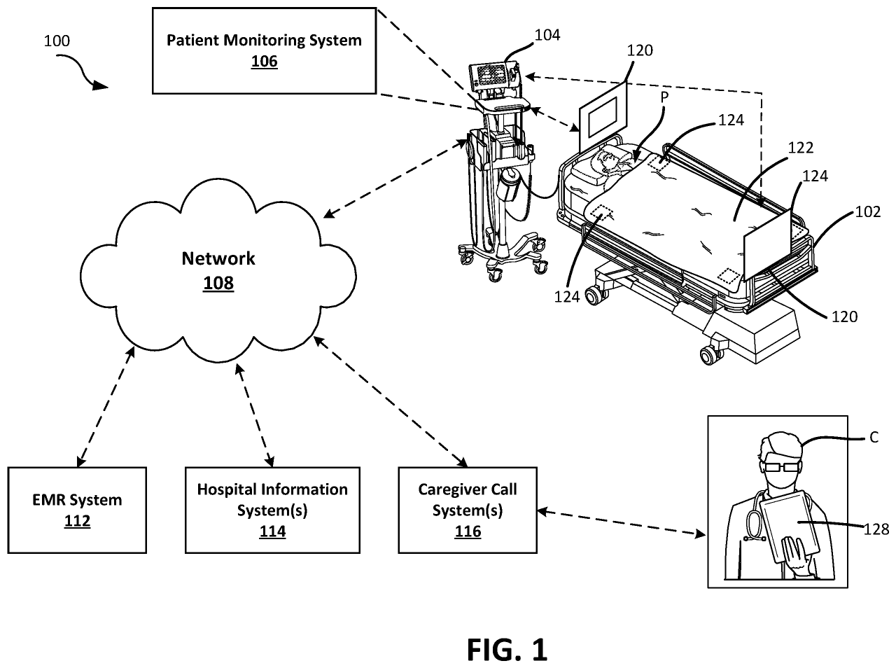

FIG. 1 is a schematic diagram illustrating an example system 100 for predicting patient bed exit. The system can be implemented, for example, at a hospital, clinic, or other healthcare facility. Patients that are at risk of falling should have caregiver assistance when getting out of bed. The system 100 operates to detect movements of a patient indicating that the patient is about to exit the bed to stand up. The system 100 can alert caregivers when a patient with high fall risk is about to exit his or her bed unattended.

One of the earliest and most prevalent signs of an upcoming bed exit is removal of covers. This often occurs even before a patient sits up or begins to shift his or her weight in preparation to get out of bed. A patient typically uses his or her feet to kick off the covers. In some instances, the patient uses his or her hands instead of or in addition to kicking to remove the covers. The term “covers” as used herein includes refers to a piece of cloth or fabric used as a body covering. The term “covers” can refer to one or more of a blanket, a sheet, a duvet, a comforter, or a quilt.

The embodiments described herein use sensors to detect movement of a patient's feet, covers on the patient bed, or both. The sensors could be one or more of Radio-Frequency Identification (RFID) sensors (tags), infrared motion detection, video motion detection, accelerometers, and load sensors in the bed. An algorithm generated from training data obtained in controlled experiments is used to analyze the sensor information to determine when patient movements indicate that covers are being removed by a patient in a bed.

In the example of FIG. 1, the system 100 for predicting patient bed exit includes a patient bed 102 in communication with a patient monitoring computing device 104. A patient monitoring system 106 operates on the patient monitoring computing device 104. The patient monitoring computing device 106 communicates via a network 108 with other computing systems including an electronic medical record (EMR) system 112, a hospital information system 114, and a caregiver call system 116.

The patient bed 102 operates to provide a surface for a patient P to rest upon while under medical care. In some embodiments, the patient bed 102 is equipped with one or more RFID readers 120. The RFID readers 120 can be configured to communicate with a network enabled smart bed 102, a patient monitoring computing device 104, or through the network 108 to other computing systems such as an EMR system 112.

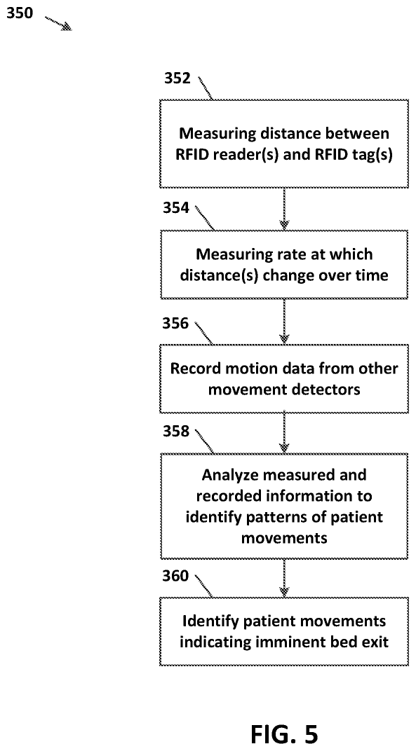

The patient bed 102 is equipped with a blanket 122 to cover the patient P. The blanket 122 includes one or more RFID sensors 124. In the example of FIG. 1, four RFID sensors 124 are embedded in the blanket 122 proximate to each of the four corners of the blanket. The RFID sensors 124 send signals that are detected by the RFID antennas 120. Movement of the RFID sensors 124 relative to the RFID readers 120 is analyzed to determine if the patient P is moving in a way that indicates that the patient P is getting out of the bed 102. This process is described in greater detail with respect to FIG. 5.

In some embodiments, the patient bed 102 is a smart bed equipped with a memory device and a processing device. The smart bed can include various functionalities to monitor a patient, entertain a patient, and make a patient more comfortable. In some embodiments, the patient bed 102 is in communication with one or more patient monitoring devices via wireless or wired connections. In some embodiments, the patient bed 102 includes load sensors and/or motion sensors to monitor patient movements on the bed. One example of a smart hospital bed is the Advanta™ 2 Med Surg Bed manufactured by Hill-Rom of Batesville, Ind.

The RFID sensors 124 function in conjunction with an RFID reader 120 to communicate via radio frequency signals. The RFID sensors may also be referred to as chips, tags, or transponders. RFID sensors generally include an integrated circuit, a means of collecting power, and an antenna. The antenna receives and transmits radio-frequency signals. The integrated circuit the stores and process information. The integrated circuit also functions to modulate and demodulate radio-frequency signals. The RFID sensors also includes a means for collecting power from the RFID reader. The RFID readers may also be referred to as RFID interrogators or antennas.

The RFID readers 120 transmit encoded radio signals to interrogate the RFID sensors 124. In response, the RFID sensors send their identification and other information such as a unique tag serial number. In some embodiments, the RFID readers are active readers and the RFID sensors are passive tags. Generally, the RFID readers are in a fixed location with an interrogation zone on the patient bed. This reduces the likelihood of accidentally communicating with RFID sensors of other patients.

In some embodiments, more than one RFID reader 120 is used to validate direction of movement of one or more RFID sensors 124. In some embodiments, multiple RFID sensors may be needed to accurately detect movement, particularly if there is only one RFID reader. In some embodiments, 13.56 MHz RFID sensors are used. In some embodiments, there are at least two RFID sensors placed apart from one another on a patient. In some embodiments, there are four RFID sensors positioned proximate to each of four corners of a blanket. In some embodiments, at least one RFID sensor is embedded in a sock worn by the patient. In some embodiments, the RFID sensors are flimsy, inexpensive and are integrated into disposable sheets. In other embodiments, the RFID sensors are more sturdy and expensive in order to withstand washing in reusable blankets and sheets.

The patient monitoring computing device 104 operates to receive and record data for a particular patient from one or more patient monitoring devices. The patient monitoring devices are in communication with the patient monitoring computing device 104 through a wired or wireless connection. Examples of patient monitoring devices include heart rate monitors, pulse oximeters, etc. In some embodiments, the patient monitoring devices can include RFID sensors 124 and RFID readers 120 as well as the patient support system (bed) itself 102.

In some embodiments, the patient monitoring computing device 104 includes a processor and memory device. The memory device can include instructions for the processor to analyze data received from patient monitoring devices. In some embodiments, the memory device can also store patient data locally. The patient monitoring computing device 104 can include a display with a user interface that allows a caregiver to easily access patient data. In some embodiments, patient monitoring computing device 104 communicates patient data to one or more of the patient monitoring system 106, EMR system 112, hospital information system 114, and caregiver call system 116 through the network 108. The patient monitoring computing device 104 can also include one or more input devices such as a keyboard, mouse, or touchscreen that receives input from a caregiver or other user.

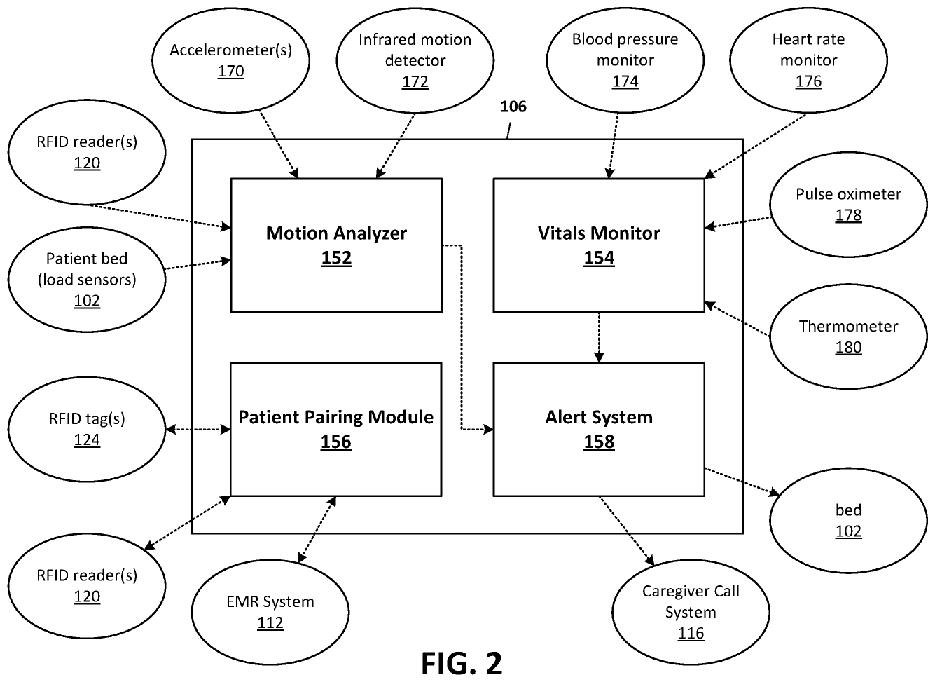

The patient monitoring system 106 operates on the patient monitoring computing device 104. In some embodiments, the patient monitoring system 106 is hosted on a remote server that is accessed by the patient monitoring computing device 104 through the network 108. The patient monitoring system 106 is described in greater detail in FIG. 2.

The network 108 operates to mediate communication of data between network-enabled computing systems. In various embodiments, the network 108 includes various types of communication links. For example, the network 108 can include wired and/or wireless links, including cellular, Bluetooth, ultra-wideband (UWB), 802.11, ZigBee, and other types of wireless links. The network 108 can include one or more routers, switches, mobile access points, bridges, hubs, intrusion detection devices, storage devices, standalone server devices, blade server devices, sensors, desktop computers, firewall devices, laptop computers, handheld computers, mobile telephones, vehicular computing devices, and other types of computing devices.

The electronic medical record (EMR) system 112 operates to record information relevant to the medical history of each patient. Examples of information that might be stored in a patient's EMR includes lab results, surgical history, family medical history, current medications, and previous medical diagnoses. A patient's fall risk score (as determined by e.g. Morse Fall Scale, Johns Hopkins Fall Risk Assessment Tool, etc.) or sub-score (as determined by Get Up and Go test) are other pieces of information that could be added to an EMR. Examples of electronic medical records systems 112 include those developed and managed by Epic Systems Corporation, Cerner Corporation, Allscripts, and Medical Information Technology, Inc. (Meditech).

The hospital information systems 114 operate to record, store, and communicate information about patients, caregivers, and hospital facilities. Hospital information systems 114 general handle administrative information for a hospital or clinic. Examples of hospital information systems 114 include admit/discharge/transfer (ADT) systems, laboratory information systems (LIS), and clinical decision support (CDS) systems.

The caregiver call systems 116 operate to generate alerts that are triggered by one or more rules. The alerts are disseminated to caregivers that need to perform critical tasks. The alerts can be generated based on data from the vital signs monitoring devices or updates to patient information that are received at the EMR system 116. As an illustrative example, patient fall risk scores, when above a predetermined threshold, trigger an alert from caregiver call system 118 that is sent to a computing device 128 associated with a caregiver C so that the caregiver is notified of the need to perform critical tasks based on the patient's fall risk. In the example of FIG. 1, the caregiver C is a nurse operating a tablet computing device 128. Other examples include smartphones, desktop computers, laptops, pagers, and other network enabled devices. In some embodiments, the alert is delivered in any suitable form, including audible, visual, and textual such as a message on a display or a pager message.

FIG. 2 is a more detailed schematic diagram of the patient monitoring system 106 of FIG. 1. In some embodiments, the patient monitoring system 106 operates on the patient monitoring computing device 104. In other embodiments, the patient monitoring system 106 operates on a remote server that is in communication with one or more patient monitoring devices. In the example of FIG. 2, the patient monitoring system 106 includes a motion analyzer 152, a vitals monitor 154, a patient pairing module 156, and an alert system 158.

The motion analyzer 152 operates to receive data from one or more devices that record patient movements. For example, in some embodiments, the motion analyzer 152 receives data from an RFID reader 120 about how far away one or more RFID sensors are from the RFID reader and whether the RFID sensors are moving. The motion analyzer 152 analyzes the data to discern particular patterns of movement indicative of a patient preparing to exit a bed. One such pattern of movement is associated with a patient removing the covers of a bed. RFID sensors embedded in a blanket change their distance from an RFID reader at an acceleration that is consistent with a patient removing the blanket in preparation to get out of bed. In some embodiments, the motion analyzer 152 receives signals based on RFID sensors placed in a patient's sock.

The motion analyzer 152 can receive data from other devices associated with a patient bed. For example, load sensors in a bed 102 can record changes in the weight present on the bed. Multiple load sensors can indicate shifts in weight as well. The load sensors can detect patient movements that are analyzed by the motion analyzer 152 to determine that a patient is about to get out of bed 102. In some embodiments, the load sensors are used in conjunction with RFID sensors to confirm that a patient is preparing to exit a bed. Other devices that can capture patterns of patient movement include infrared motion detectors 172, video motion sensors, and accelerometers 170 placed on the patient.

The vitals monitor 154 operates to receive and analyze data from one or more vitals monitoring devices associated with a patient. In some embodiments, the vitals monitoring devices monitor one or more of a patient's body temperature, blood pressure, heart rate, blood oxygen level, and respiration rate. As shown in FIG. 2, the vitals monitor 154 can receive data from one or more of a blood pressure monitor 174, a heart rate monitor 176, a pulse oximeter 178, and a thermometer 180. Other vitals monitors are possible. In some embodiments, the vitals monitor 154 operates to analyze data received from vitals monitoring devices to determine when an alert needs to be issued for the patient. The alert can be communicated to a caregiver through, for example, the caregiver call system 116.

The patient pairing module 156 operates to set up a patient support system 102 with accompanying monitoring devices and computing devices for a particular patient. The patient's ID and EMR is associated with the patient monitoring computing device 104 to ensure that the correct patient information is displayed and that the data being recorded by monitoring devices is recorded to the correct patient EMR in the EMR system 112. Any motion detecting devices are paired to the patient monitoring computing device 104 via wired or wireless connections. In some embodiments, the patient pairing module 156 ensures that RFID sensors 124 in a patient's covers 122 or socks are properly paired with the RFID readers 120 at the patient's bed 102 as well as the patient monitoring computing device 104. Any RFID sensors 124 are thus associated with the correct patient.

The alert system 158 operates to communicate alarms or alerts to computing systems in communication with the patient monitoring computing device 104 or patient bed 102. For example, the alert system 158 can communicate alerts to caregiver call systems 116 to notify caregivers of the imminent risk of a patient fall. The alerts can be disseminated to a status board or caregiver mobile devices. The alert system 158 can also activate an alert response at the patient bed 102.

If the patient bed 102 is equipped with safety devices to mitigate falls, those devices can be automatically activated to provide one or more fall risk mitigation actions. For instance, some patient beds are equipped with side rails that can automatically be locked and/or moved up or down (e.g., motorized). In such an alert situation, the side rails can be locked (if already in the up position) and/or moved to an up position to further minimize the likelihood of the patient exiting the patient bed 102.

The alert system 158 can also communicate a visual or audible alert at the patient monitoring computing device 104 or bed 102. In some embodiments, the alert at the patient bed instructs the patient to stay in bed or to wait for a caregiver to arrive. This alert could be a voice command delivered over a speaker at the patient bed 102 or placed elsewhere near the patient bed. In other examples, alerts are provided to the caregiver as well, such as at a central station and/or mobile device of the caregiver.

In the example of FIG. 1, when the patient P is removing the covers 122, the RFID sensors (or tags) move closer to or further away from an RFID reader 120. The RFID reader 120 communicates the distance and speed at which the distance is changing to the patient monitoring computing device 104, where the motion analyzer 152 processes the data to determine whether the patient's patterns of movement indicate that the patient is about to get out of the bed. When such patterns of movement are recognized, this is communicated to the alert system 158. The alert system 158 determines which other computing systems need to be notified for that particular patient P. This determination can be informed by data received from the vitals monitor 154 as well as the patient's EMR. The alert system 158 can communicate alerts to a caregiver call system 116 through the network 108 as well as other hospital information systems 114. In turn, the caregiver call system 116 disseminates alerts to one or more caregiver computing devices 128 to notify particular caregivers C responsible for the patient P. At the same time, the alert system 158 communicates an order to the patient bed 102 to project a visual warning on the floor next to the bed so that the patient is reminded not to get out of bed unattended. Any caregivers passing by the patient's bed will notice that the patient should not be getting out of bed unattended and can come to aid the patient.

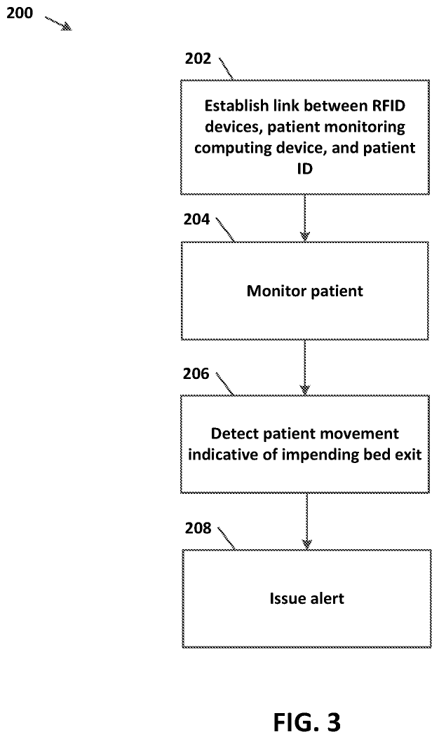

FIG. 3 is a flow chart illustrating an example method 200 of monitoring a patient to mitigate a risk of falling. In some embodiments, one or more aspects of this method are performed by the patient monitoring system 106 of FIGS. 1 and 2.

At operation 202, a link is established between the RFID devices (readers and sensors), patient monitoring computing device, and patient identifier. In some embodiments, this is performed by the patient pairing module 156 of FIG. 2. This occurs when the patient is set up in a bed 102 to be monitored by a patient monitoring computing device 104. The linking process ensures that the correct patient data is retrieved from the EMR system and that any data recorded on patient monitoring devices (including the bed itself) are recorded with the correct patient's EMR. Further, this step ensures that any RFID sensors on the patient or the patient's blanket are being read by the correct RFID reader associated with the patient's bed. It is possible that without proper pairing, a RFID reader at a first patient's bed could receive signals from RFID sensors on a second patient, if the second patient is within range of the RFID reader.

At operation 204, the patient is monitored using the patient monitoring computing device 104 in communication with vitals sign monitoring devices and motion detecting devices. In some embodiments, the motion detecting devices include at least one RFID reader 120 and at least one RFID sensor 124 embedded in covers placed over the patient. In some embodiments, patient movement data is analyzed by the motion analyzer 152 of FIG. 2. In some embodiments, vital signs are monitored by the vitals monitor 154.

At operation 206, patient movements indicative of an impending bed exit are detected. In some embodiments, this operation is performed by the motion analyzer 152. When such movements are detected, the motion analyzer 152 communicates that information to the alert system 158. In some embodiments, the patient movements are determined based on readings of distance between RFID sensors embedded in a patient's blanket or sock and an RFID reader mounted on or near the patient's bed. Changes in that distance can indicate that a patient is removing the covers in preparation to get out of bed. Alternatively, or in addition to the RFID readings, other motion detection methods can be used. For example, infrared motion detection, load sensors in the bed, and computer vision can also detect patient movements. Algorithms in the motion analyzer 152 determine which patterns of movement are most likely to precede a patient getting out of bed.

At operation 208, an alert is issued indicating that the patient is at risk of falling. In some embodiments, this operation is performed by the alert system 158 of FIG. 2. Alerts can be communicated to caregivers to notify them of an impending risk of a patient fall. Alerts can also be communicated to a patient monitoring computing device 104 near the patient's bed that can automatically implement fall risk mitigation actions.

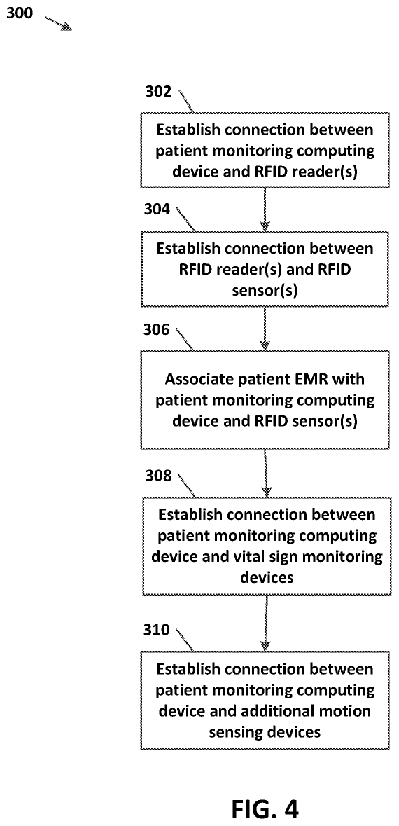

FIG. 4 illustrates a flow chart of a more detailed example method 300 of setting up a patient monitoring system with patient movement detecting devices. In some embodiments, this method 300 is performed by the patient pairing module 156 of FIG. 2.

At operation 302, a connection is established between a patient monitoring computing device and at least one RFID reader positioned proximate a patient bed. The connection can be a wired or wireless connection. In some embodiments, the RFID reader 120 is paired to the patient monitoring computing device 106 through a short-range wireless communication connection such as Bluetooth. In some embodiments, the RFID reader 120 is connected to the patient bed 102, which in turn communicates with the patient monitoring computing device 106.

At operation 304, a connection is established between the RFID reader and at least one RFID sensor placed on a patient in the patient bed. In some embodiments, the RFID sensor 124 is embedded in one or more of a blanket, a sock, a bracelet, and an anklet placed on the patient such that the RFID sensor 124 moves in a predictable manner when the patient removes the covers of the bed to exit the bed.

At operation 306, the patient's EMR is paired to the patient monitoring computing device and associated RFID devices. In some embodiments, the patient monitoring computing device 106 communicates with an EMR system 112 to access a patient's EMR when prompted by a caregiver. The RFID reader 120 transmits information about the status of connected RFID sensors 124 to the patient monitoring computing device 106, which then can record information to the patient's EMR.

At operation 308, connections between vital signs monitoring devices and the patient monitoring computing device are established. In some embodiments, the vitals monitor 154 of the patient monitoring computing device 106 receives data from one or more of an infrared motion detector 172, blood pressure monitor 174, heart rate monitor 176, pulse oximeter 178, and thermometer 180. The vital signs monitoring devices can be connected to the patient monitoring computing device 106 via wired or wireless connections. For example, the vital signs monitoring devices could plug into the patient monitoring computing device 106 or to the patient bed 102. In other examples, the vital signs monitoring devices could communicate with the patient monitoring computing device 106 via Bluetooth, Wi-Fi, NFC, etc.

At operation 310, connections between additional movement detecting devices and the patient monitoring computing device are established. Other movement detecting devices can include infrared motion sensors and video motion sensors that can communicate via wired or wireless connections.

FIG. 5 is a flow chart illustrating a more detailed example method 350 of monitoring a patient to mitigate falls. In some embodiments, this method 350 is performed by the patient monitoring system 106 of FIGS. 1 and 2.

At operation 352, the distance between one or more RFID readers 120 and one or more RFID sensors 124 is measured. In some embodiments, this operation is performed by the motion analyzer 152 of FIG. 2. Measurements of the distance between each RFID reader 120 and RFID sensor 124 at a patient bed 102 is measured over time. Changes in the distance indicates that the patient or a blanket 122 covering the patient has moved. The changes in distance can be used to infer movement of the patient.

At operation 354, the rate at which the distance between the RFID readers 120 and RFID sensors 124 changes over time is measured. Slow changes in the distance between RFID readers 120 and RFID sensors 124 embedded in the covers 122 may mean that a blanket is simply slipping down or a patient is getting warm. However, quick changes in the distance between RFID sensors and RFID readers on a patient bed could indicate that the patient is removing the covers in preparation for exiting the bed. Also, in situations where there are multiple RFID readers 120 and multiple RFID sensors 124, the particular combinations of tags and readers and how the distance change can be analyzed to infer particular types of movement that occur when a patient is preparing to exit a bed 102.

At operation 356, motion data from other movement detectors is optionally recorded. In some embodiments, additional data can be used to aid in assessing whether a patient is about to exit a bed. For example, the motion analyzer 152 could receive load sensor data from the patient bed 102 to determine how the patient's weight is shifting on the bed. In another example, an accelerometer 170 in a wristband worn by the patient could record movements consistent with a patient removing the covers. An infrared motion detector 172 or video motion detector could record patient movements that can be analyzed to determine if a patient is about to get out of bed.

At operation 358, the measured and recorded information is analyzed to identify patterns of patient movements. In some embodiments, this operation is performed by the motion analyzer 152. In some embodiments, the motion analyzer 152 employs a machine learning generated model to analyze patient movement data. The machine learning model is generated by training a machine learning algorithm with patient movement data from controlled experiments. Patient bed exits are identified in the experimental data and the corresponding patient movements are identified by the algorithm. The resulting machine learning model is used to classify patterns of patient movements measured from RFID sensors and other motion detectors.

At operation 360, patient movements are identified that indicate imminent bed exit. In some embodiments, the motion analyzer 152 operates to identify the patterns of patient movements indicative of imminent bed exit using the machine learning model. When patient movements indicating imminent bed exit are detected, a message can be communicated to the alert system 158 of FIG. 2 for processing.

In some embodiments, the algorithm for detecting imminent bed exit relies upon measurements of distance between RFID sensors and RFID readers at the patient's bed. One example of such an algorithm is:

change in distance between tag and reader/time=rate of distance change
where rate of distance change>x indicates patient is removing covers

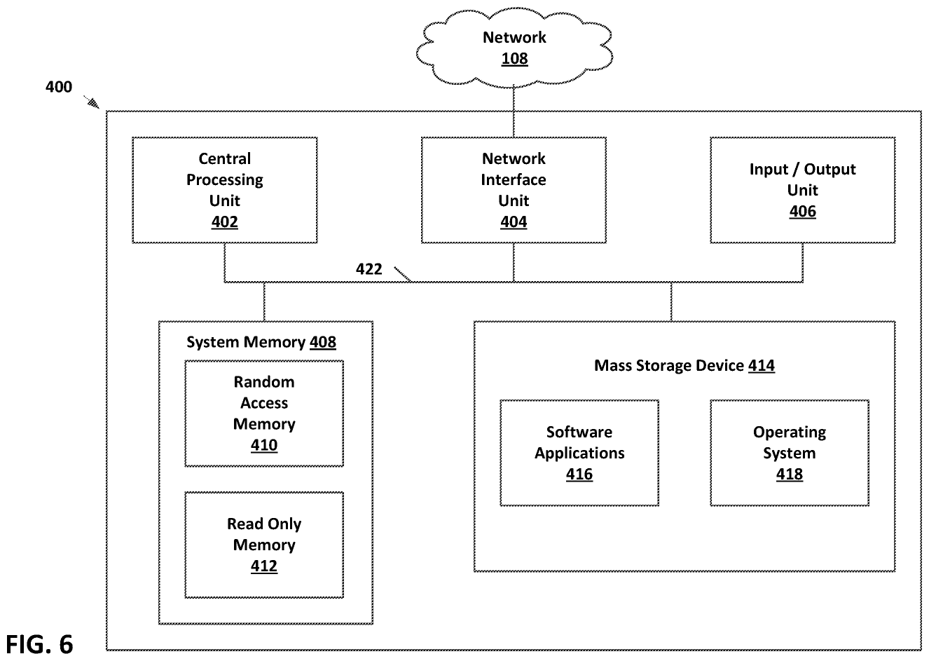

FIG. 6 is a block diagram illustrating an example of the physical components of a computing device 400. The computing device 400 could be implemented in various aspects of the system 100 for predicting bed exit. Components of the computing device 400 can also be incorporated into other devices described herein, such as the patient monitoring computing device 104 or a computing device integrated into the bed 102.

In the example shown in FIG. 6, the computing device 400 includes at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system that contains the basic routines that help to transfer information between elements within the computing device 400, such as during startup, is stored in the ROM 412. The computing system 400 further includes a mass storage device 414. The mass storage device 414 is able to store software instructions and data such as movement data received from the RFID readers 120 or patient bed 102.

The mass storage device 414 is connected to the CPU 402 through a mass storage controller (not shown) connected to the system bus 422. The mass storage device 414 and its associated computer-readable storage media provide non-volatile, non-transitory data storage for the computing device 400. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 402 can read data and/or instructions. In certain embodiments, the computer-readable storage media comprises entirely non-transitory media.

Computer-readable storage media includes volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 400.

According to various embodiments, the computing device 400 can operate in a networked environment using logical connections to remote network devices through a network 106, such as a wireless network, the Internet, or another type of network. The computing device 400 may connect to the network 108 through a network interface unit 404 connected to the system bus 422. It should be appreciated that the network interface unit 404 may also be utilized to connect to other types of networks and remote computing systems. The computing device 400 also includes an input/output controller 406 for receiving and processing input from a number of other devices, including a touch user interface display screen, or another type of input device. Similarly, the input/output controller 406 may provide output to a touch user interface display screen or other type of output device.

As mentioned briefly above, the mass storage device 414 and the RAM 410 of the computing device 400 can store software instructions and data. The software instructions include an operating system 418 suitable for controlling the operation of the computing device 400. The mass storage device 414 and/or the RAM 410 also store software instructions, that when executed by the CPU 402, cause the computing device 400 to provide the functionality discussed in this document. For example, the mass storage device 414 and/or the RAM 410 can store software instructions that, when executed by the CPU 402, cause the computing system 400 to analyze movement data received from motion detectors at a patient's bed.

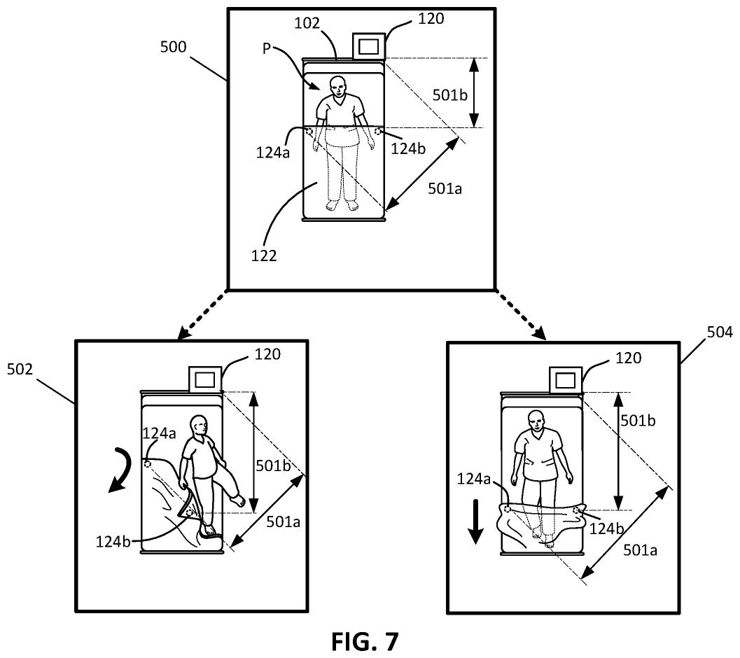

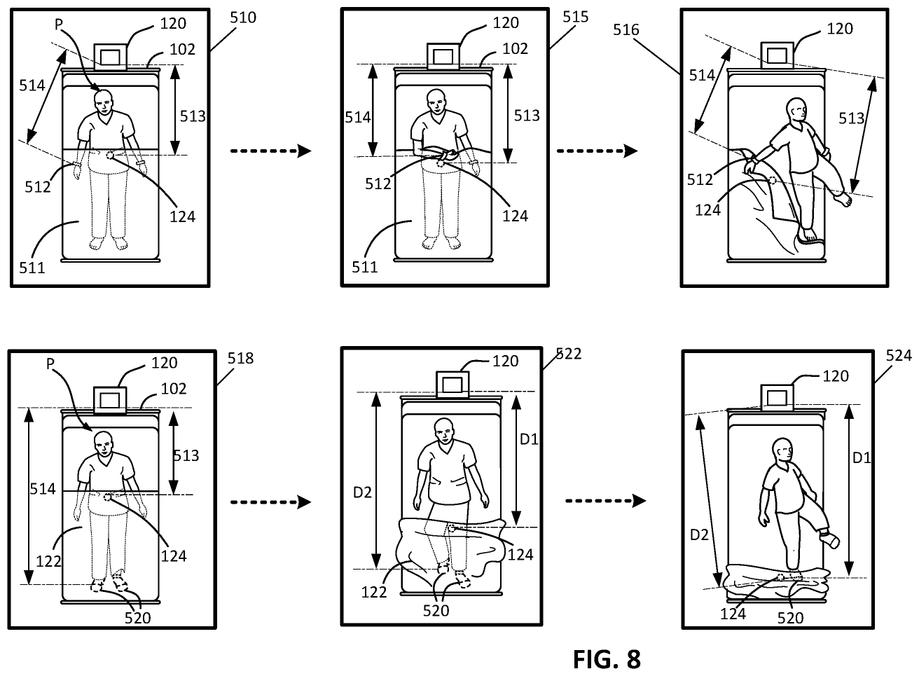

FIGS. 7 and 8 illustrate examples of how patient movements could be recorded with RFID devices. FIG. 7 illustrates examples of patient movements when a patient P is lying on a bed 102 under covers 511 having two RFID sensors 124 embedded therein. An RFID reader 120 is positioned at the head of the bed and two RFID sensors 124 are embedded in the top of the covers 511, nearest the head of the bed 102.

In the first view 500, the patient P is lying under a blanket 122 on the bed 102. The RFID sensors 124 are approximately equal distances from the RFID reader 120. The distance 501a between the first RFID sensor 124a and the RFID reader 120 is greater than the distance 501b between the second RFID sensor 124b and the RFID reader 120.

In the second view 502, the patient P has grasped one corner of the blanket 122 and moved it to the opposite side of the bed to remove the blanket. This movement has shifted the second RFID sensor 124b further from the RFID reader 120 so that the distance 124b is greater. The first RFID sensor 124a has remained the same distance 124a from the RFID reader 120.

These changes in distance between the RFID sensors 124 and RFID reader 120 occur quickly enough to indicate that the patient is deliberately moving the blanket 122. The patient monitoring system 106 would analyze these changes in distance and determine that the patient is about to get out of bed. In this example, if load sensors were in the bed reading changes in load, they would indicate a shift in weight as the patient sat up. This would supplement the RFID data to confirm that the patient is preparing to get out of bed. In some instances, the RFID sensors alone might provide ambiguous indications about the patient's movements, but additional motion detecting devices could confirm the movements as being precursors to bed exit. For instance, a video motion detector could confirm that the patient is moving to exit the bed.

The third view 504 shows another way that the patient P might move to remove the blanket 122 in preparation for exiting the bed 102. Here, the patient is still lying down, but is kicking off the blanket 122. Both RFID sensors 124a, 124b are quickly moved away from the RFID reader 120. The patient monitoring system 106 would analyze this rapid increase of distances 501a, 501b and identify it as being consistent with an imminent patient bed exit. The patient monitoring system 106 would issue an alert to nearby caregivers to prompt them to come aid the patient in getting out of bed.

FIG. 8 illustrates examples of patient movements on a bed 102 with RFID sensors 124 embedded in articles of clothing that the patient is wearing. In these examples, the RFID sensors 124 are in a wristband 512 or socks 520.

In the top left view 510, the patient P is lying on the bed 102. An RFID reader 120 is positioned at the center of the head of the bed. The patient is wearing a bracelet 512 with an RFID sensor embedded inside. Additionally, a single RFID sensor 124 is embedded in the top center of the covers 511. The distance 513 between the RFID sensor 124 and the RFID reader 120 is slightly less than the distance 514 between the bracelet 512 and the RFID reader 120.

In the top center view 515, the patient P is reaching with his right hand to grasp the covers 511 at his left side. As the patient P makes this movement, the RFID sensor 124 moves slightly away from the RFID reader 120 and the bracelet 512 moves slightly closer to the RFID reader 120. The RFID reader 120 also records the speed at which the bracelet 512 moves, which indicates a deliberate movement. However, without more, an alert is not triggered for the patient.

In the top right view 516, the patient P has moved his right arm back to the right side of the bed 102, pulling the covers 511 off of himself and he is starting to get off of the bed 102. The bracelet 512 has moved to the right and thus the distance 514 between it and the RFID reader 120 has increased again. Additionally, the RFID reader 120 records how quickly the bracelet 512 is moving. The RFID sensor 124 embedded in the covers 511 has moved further from the RFID reader 120, increasing the distance 513. The combination of the changes in distances as well as the speed at which those changes occurred would prompt the motion analyzer 152 to determine that the patient P is about to exit the bed 102.

In the lower left view 518, the patient P is lying in the bed 102, wearing socks 520 having RFID sensors embedded therein. While the patient is lying on the bed, the distance 514 between the RFID sensors in the socks 520 and the RFID reader 120 is about the same and does not change very much or very quickly. The distance 513 between the RFID sensor 124 in the covers 511 and the RFID reader 120 is much less than the distance 514 between the socks 520 and the RFID reader 120.

In the lower center view 522, the patient P is kicking off the covers 511. While this is occurring, the distance 514 between the socks 520 and the RFID reader 120 is fluctuating quickly. Additionally, the distance 513 between the RFID sensor 124 and the RFID reader 120 is growing larger. In some instances, this is enough for the motion analyzer 152 to determine that the patient P is attempting to exit the bed 102.

In the lower right view 524, the patient P has kicked the covers 511 completely off and is starting to exit the bed. The distance 513 between the RFID sensor 124 and the RFID reader 120 is even greater. The distance 514 between the socks 520 and the RFID reader 120 is still fluctuating. These measurements provide further information to the motion analyzer 152 to support a finding that the patient P is attempting to exit the bed 102.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A method of predicting exit of a patient support system, the method comprising:
establishing a connection between a patient monitoring computing device and at least one radio frequency identification (RFID) reader positioned proximate the patient support system;
establishing a connection between the at least one RFID reader and at least one RFID sensor associated with one or more of a blanket, a sock, a bracelet, and an anklet placed on a patient in the patient support system;
monitoring, with the patient monitoring computing device, movement on the patient support system using data from the RFID reader, the data indicating a distance between the at least one RFID sensor and the at least one RFID reader; and
determining when the data indicates that the patient is exiting the patient support structure.

2. The method of claim 1, wherein the at least one RFID reader is attached to the patient support system.

3. The method of claim 1, wherein the at least one RFID sensor is embedded in covers placed over the patient in the bed.

4. The method of claim 3, wherein the at least one RFID sensor is four RFID sensors positioned proximate to each of four corners of a blanket.

5. The method of claim 1, wherein the at least one RFID sensor is two RFID sensors positioned apart from one another.

6. The method of claim 1, wherein the at least one RFID sensor is embedded in a sock worn by the patient.

7. The method of claim 1, wherein patient movements indicating that the patient is exiting the patient support system are determined by a machine learning algorithm.

8. The method of claim 1, wherein monitoring patient movements is further based on one or more of load sensor data from the patient support system, infrared motion detection, and accelerometers positioned on the patient.

9. The method of claim 1, further comprising communicating an alert to a caregiver call system for dissemination to one or more caregiver computing devices.

10. The method of claim 1, further comprising communicating an alert to the patient support system and a fall risk mitigation action is performed automatically at the patient support system.

11. A system for monitoring patient movements on a bed, the system comprising:
a bed configured to support a patient while under medical care;
at least one RFID reader positioned proximate the bed;
two or more RFID sensors embedded in covers configured to cover a patient on the bed; and
a patient monitoring computing device comprising a processor and a memory comprising instructions that, when executed, cause the processor to operate a patient monitoring system configured to perform a series of operations comprising:
establishing a connection between the patient monitoring computing device and the at least one RFID reader;
establishing a connection between the at least one RFID reader and the two or more RFID sensors;
associating the RFID sensors with a patient at the patient monitoring computing device;
monitoring patient movements on the bed based on signals from the RFID reader measuring a distance between the two or more RFID sensors and the at least one RFID reader;
detecting patient movements indicating that the patient is exiting the bed; and
issuing an alert to a caregiver call system.

12. The system of claim 11, wherein the bed comprises one or more load sensors configured to detect patient movements.

13. The system of claim 11, further comprising one or more vitals sign monitoring devices in communication with the patient monitoring computing device.

14. The system of claim 11, further comprising an accelerometer positioned on the patient configured to detect patient movements.

15. The system of claim 11, wherein the at least one RFID reader comprises two RFID readers affixed to the patient bed at two different locations.

16. The system of claim 11, wherein patient movements indicating that the patient is exiting the bed are determined based on the speed at which the distance between the two or more RFID sensors and the at least one RFID reader changes.

17. The system of claim 16, wherein the patient movements indicating that the patient is exiting the bed are further determined based on one or more of load sensor data, accelerometer data, motion detector data, and patient EMR data.

18. The system of claim 11, further comprising activating an alert response at the bed.

19. The system of claim 11, further comprising recording patient movements in an electronic medical record (EMR) of the patient.

20. One or more computer-readable media having computer-executable instructions embodied thereon that, when executed by one or more computing devices, cause the computing devices to:
establish a connection between a patient monitoring computing device and at least one radio frequency identification (RFID) reader positioned proximate a patient bed;
establish a connection between the at least one RFID reader and at least two RFID transponders embedded in one or more of a blanket and a sock placed on a patient in the patient bed;
associate the at least one RFID transponder with the patient at a patient monitoring computing device;
monitor, with the patient monitoring computing device, patient movements on the bed based on signals from the RFID reader measuring a distance between the RFID transponders and the at least one RFID reader;
detect patient movements indicating that the patient is going to exit the bed, the patient movements being determined based on the speed at which the distance between the two or more RFID sensors and the at least one RFID reader changes; and
issue an alert to a caregiver call system.
